(19) United States
(12) Patent Application Publication
(10) Pub. No.: US 2006/0247538 A1
(43) Pub. Date: Nov. 2, 2006

(54) Noninvasive method of determining arterial wall tension and arterial segmentation by pulse transit time and pulse wave velocity

(76) Inventor: Charles L. Davis, Portland, OR (US)

Correspondence Address: Pauley Petersen & Erickson, Suite 365, 2800 West Higgins Road, Hoffman Estates, IL, 60195, US

(21) Appl. No.: 11/412,370

(22) Filed: Apr. 27, 2006

Related U.S. Application Data
(60) Provisional application No. 60/675,270, filed on Apr. 27, 2005.

(51) Int. Cl. A61B 5/02

(52) U.S. Cl. 600/481; 600/485; 600/490; 600/504

(57) ABSTRACT

A method of noninvasively obtaining a physiological parameter of a fluid vessel. A series of pressure values are applied to a region of the vessel to adjust the transmural pressure of the vessel wall. At each of the pressure values at least one of a pulse transit time and a pulse wave velocity through the region of the vessel is measured. At least one of vessel compliance and vessel segmentation is determined as a function of the pulse transit time or pulse wave velocity and the applied pressure.

### CROSS REFERENCE TO RELATED APPLICATION

This application claims priority to Provisional Patent Application Ser. No. 60/675,270, filed on 27 Apr. 2006. The co-pending Provisional Application is hereby incorporated by reference herein in its entirety and is made a part hereof, including but not limited to those portions which specifically appear hereinafter.

### BACKGROUND OF THE INVENTION

1. Field of the Invention

This invention relates to noninvasive determination of vascular functionality including arterial wall tension (compliance) and arterial segmentation, and more particularly to observing Pulse Transit Time and Pulse Wave Velocity in a region of a living subject while applying a series of pressures to the region.

2. Discussion of Related Art

Vascular functionality including Arterial Wall Tension or Compliance is an important parameter in the evaluation and treatment of vascular diseases such as hypertension and peripheral artery disease (PAD). Arterial segmentation is important in the evaluation of the autonomic control of pressure and fluid volume distribution in the vascular system.

Much of the related art has been discussed in commonly owned U.S. Pat. No. 6,749,567 and U.S. Pat. No. 7,011,631, both of which are included herein by reference in their entirety.

Pulse Wave Velocity (PWV) and Pulse Transit Time (PTT) measurements have been made in a variety of ways as disclosed in the art, such as in U.S. Pat. Nos. 4,425,920; 6,331,162; and 6,511,436.

### SUMMARY OF THE INVENTION

The present invention provides means and methods for noninvasively determining vascular functionality including the arterial wall tension or compliance and the arterial segmentation in a pulsed flow system with non-rigid wall vessels, such as found in the vascular system of living subjects. This invention makes use of noninvasive measurements of fluid pressure or fluid volume changes at separate locations along the path of fluid pulse wave propagation in the region of the living subject. Fluid pulse wave velocity and fluid pulse transit time are measured through a region of vessels as the transmural pressure of the vessels are manipulated by changes in internal pressure or external pressure of the vessel. When linear pressure application is increased from zero to a suprasystolic pressure and then released over time until the applied pressure is again zero, the resulting changes in pulse wave velocity and pulse transit time data versus pressure can be used to determine the characteristic changes in wall tension (or compliance) of the vessels. Furthermore, the discontinuities in the relationship between pulse wave velocity, pulse transit time and pressure can be used for segmentation of the vessels into their serial identity segments, as demonstrated in U.S. Pat. No. 6,749,567. Sample graphs of data collected on swine and humans are shown in FIGS. 22a and 22b.

The primary application of this invention is the noninvasive evaluation of vascular disease in humans and animals, but the methods disclosed herein will apply to any pulsed flow system with non-rigid wall vessels.

The general advantages of noninvasive measurement of pulse wave velocity and pulse transit time compared to current practices of invasive measurement include a reduced risk of contracting blood-borne diseases for care givers and patients, elimination of patient risk of infection, clotting, and blood vessel damage, early warning of impending Shock condition that will help save lives now lost, lower cost of procedures, less time consumption per procedure for clinicians, increased speed and repeatability of measurements that improves the reporting of results, and reduction in patient discomfort.

The general object of the invention can be attained, at least in part, through a method of noninvasively obtaining a physiological parameter of a fluid vessel by applying a series of pressure values to a region of the vessel to adjust the transmural pressure of the vessel wall, measuring at each of the pressure values at least one of a pulse transit time and a pulse wave velocity through the region of the vessel, and determining at least one of vessel compliance and vessel segmentation as a function of the pulse transit time or pulse wave velocity and the applied pressure.

Specific users of the present invention may include physician offices, ambulances, trauma/emergency care centers, military field operations, surgery, hemodialysis, blood banks, and Ob/Gyn practitioners.

### BRIEF DESCRIPTION OF THE DRAWINGS

For a better understanding of the invention, and to show how the same may be carried into effect, reference will now be made, by way of example, to the accompanying drawings, in which

FIG. 1a depicts a vascular loop from the left ventricle to the right atrium of the heart showing the distribution of large arteries, small arteries, arterioles, capillaries, venules, small veins, and large veins. It further depicts the boundary conditions which separate these different vessel types.

FIG. 1b depicts three views of a blood vessel and the relationships between the applied pressure and the volume behavior of the vessel. The ‘Longitudinal View’ depicts the relationship of the pressure application to the pulse sensors adjacent to the pressure affected blood vessel area. The “Axial View’ depicts the relationship of the internal and external pressures in the system as they determine the transmural pressure across the wall of the vessel. The ‘Crushed or Deflated View’ depicts the vessel appearance from an axial view when the vessel has been fully crushed or deflated.

FIG. 1c depicts various inflation and deflation modes of pressure application in the pressure applicator.

FIG. 1d depicts the types of timing markers that can be used to determine pulse transit timing and pulse wave velocity measurements.

FIGS. 2a and 2b depict equivalent circuit models of the body given by parallel conductor theory.

FIG. 3 is a graphical representation of the fluid admittances in a subject's limb, all of which are proportional to fluid volumes, showing how they combine to form the total volume under the pressure cuff.

FIGS. 4 and 5 are illustrations of the impedance sensor in three planes and indicates the various measuring channels.

FIG. 6 is a sectional view of a subject's arm showing the cuff and impedance sensor applied.

FIG. 7 is a block diagram of one embodiment of the present invention, including a pressure sensor, an inflatable cuff pressure generator, a bioimpedance fluid volume sensor, and a monitor.

FIGS. 8 through 13 are enlargements of the various other functional blocks shown in the block diagram of FIG. 7 and depict the internal functions of these blocks.

FIGS. 14 and 15 are graphs depicting changes in pressure in the cuff over time, and indicate how various pressures are measured.

FIG. 16 is a graph of oscillometric pulse pressures versus cuff pressures.

FIGS. 17 through 20 are graphs of values acquired using the apparatus shown in FIG. 7, and illustrate a methodology for subtracting non-blood admittances, leaving only the basal admittance of the blood in the artery.

FIG. 21 is a graph depicting the admittance against cuff pressure and indicating how the ratio of the pulsatile change in admittance and the absolute value of admittance are calculated.

FIGS. 22a and 22b are graphs depicting the change in pulse transit time along with associated pressure changes for a young healthy adult swine and an older hypertensive adult male.

FIG. 22c is a graph representing segmentation by slope of t vs. pulse number zero crossings.

FIG. 22d is a graph representing segmentation by slope of t vs. pressure and pulse number zero crossings

### DETAILED DESCRIPTION

The present invention provides means and methods for noninvasively determining changes in the vessel wall tension or compliance of the vessel, and the vessel segmentation in a pulsed flow system with non-rigid wall vessels such as found in the vascular system of living subjects. The invention will be described herein below with reference to arterial stiffness and vascular segmentation. It will be appreciated that the sphere of the present invention need not be limited strictly to vascular blood systems. The present disclosure will demonstrate the measuring of the pulse transit time and pulse wave velocity through a region of vasculature using time varying volume changes or time varying pressure changes for measuring these parameters. It will be appreciated that any means of measuring time varying changes of the vascular bed such as tonometry, optical transmission and reflection, ultrasound, bioimpedance, or any noninvasive pressure or volume sensing means for the purpose of determining pulse transit time or pulse wave velocity in a region of vessels will be sufficient for implementation of this invention and are included herein. Means for changing internal and external coextensive pressures in the region of vessels by modification of internal vascular hydrostatic pressure by change of elevation of the body region relative to the level of the heart or use of external pressure application devices are included herein.

An important feature of this invention is the ability to measure pulse transit times and pulse wave velocity over short, non-bifurcating, lengths of the vasculature. Until this invention, methods of PTT and PWV measurements were taken over longer lengths of vasculature, (for example, Asmar, U.S. Pat. No. 6,511,436), and typically incorporated pulse wave transmission pathways that travel from the thorax to a peripheral limb. These transmission pathways often involve bifurcations of vessels along the pathway which induce the effects of peripheral resistance and wave reflections into the PTT and PWV measurements. When PTT and PWV measurements are made over short vascular pathways, the effects of peripheral vascular resistance are minimized or eliminated from the measurement. Short vascular pathways for measurement of arterial pulse wave behaviors can typically be made on the limbs of a subject as well as the neck or fingers.

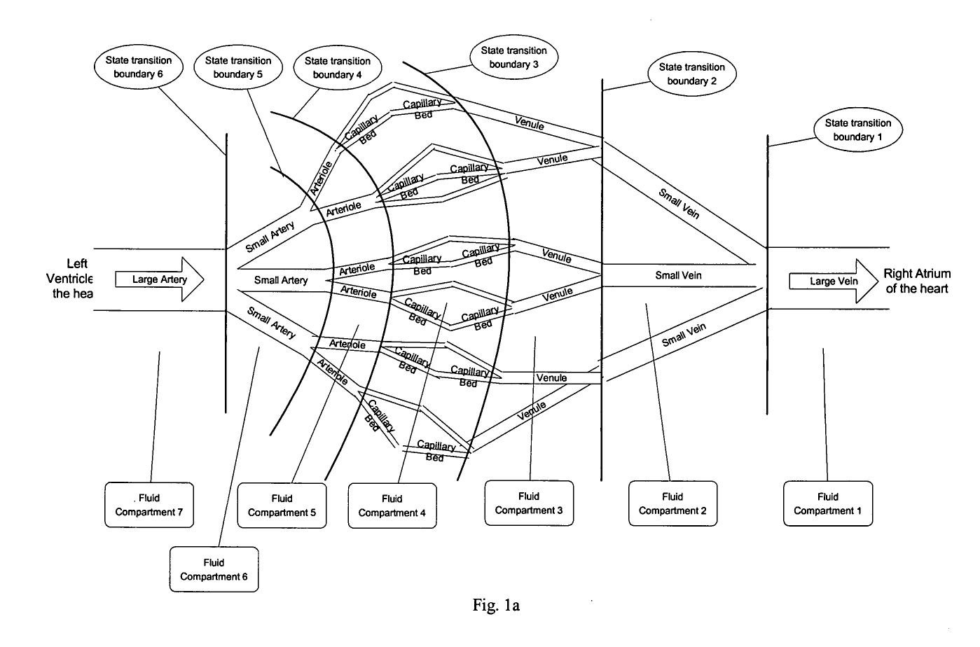

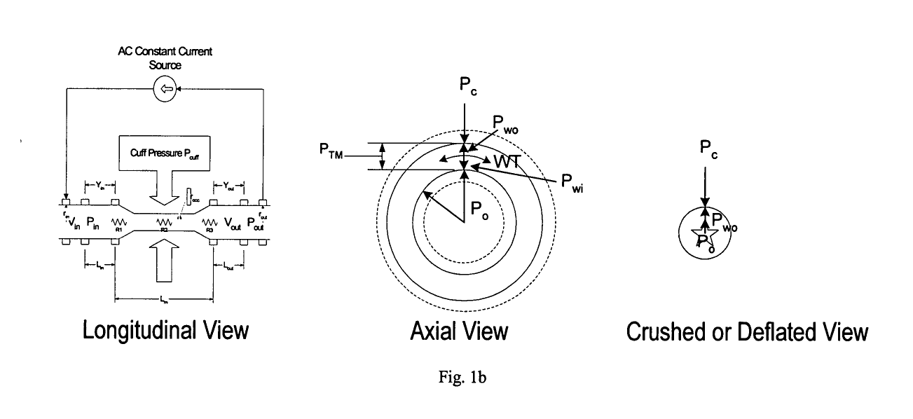

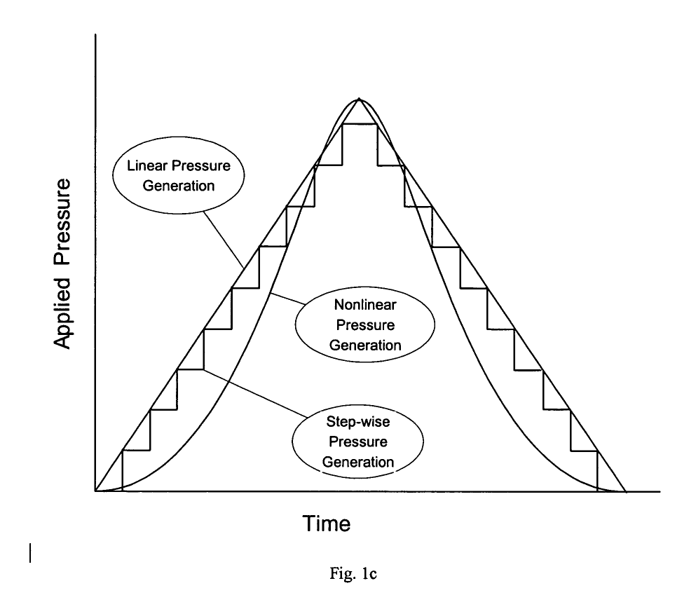

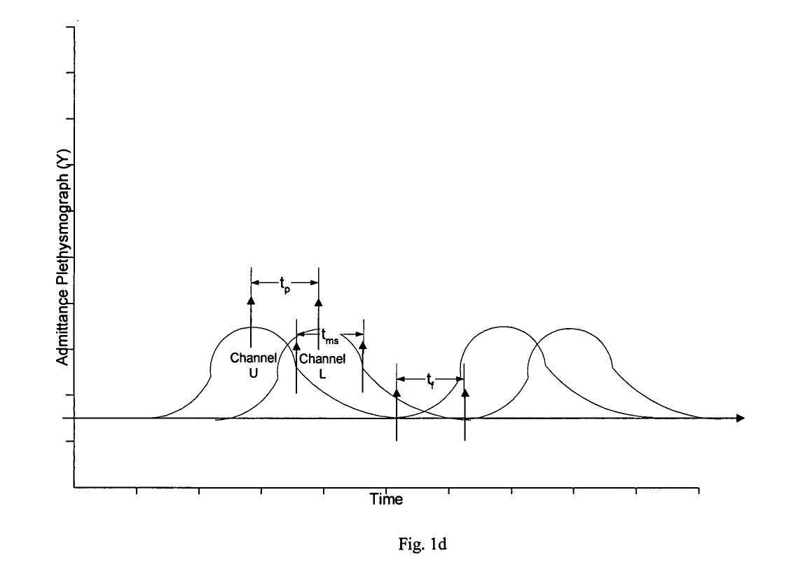

External pressure generation may be applied in a variety of modes as depicted in FIG. 1c. However, for purposes of the exemplary embodiment, the linear pressure generation mode is preferred.

An important benefit of either applying pressure to the measured region of vasculature (Pc, FIG. 1b) or changing the internal hydrostatic pressure of the region of vasculature (Po, FIG. 1b) while making the pulse transit time or velocity measurement through that region of vessels is that the change in either the external or internal pressures causes equivalent changes in the transmural pressure (Pₜₘ, FIG. 1b) across the wall of the vessel thereby changing the effective elasticity of the vessel. A change in the transmural pressure of the artery wall causes a concomitant change in the elasticity, compliance, or wall tension of the arterial wall. The change in effective compliance of the vessel due to the change in artery wall transmural pressure causes a change in the velocity of the pulse wave moving through that region. This phenomena results from the fact that changes in the loading of the artery walls in the measured region, causes changes in the effective compliance of the vessel wall. This unloading of the arterial walls leads to an effective increase in the compliance of the artery and thus an increase in the apparent compressibility of the blood. This results in a slowing of the propagating volume and pressure waves as they pass through the measured region. When using a linear pressure mode, the relationship of change in PTT or PWV versus applied pressure will illustrate regions of linear change in PTT versus pressure as well as regions of discontinuity in PTT change versus pressure change. These areas of discontinuity are indicative of filling pressures of the different types of arterial vessels in the measured region of the body. Therefore, this method can be used to determine pressure segmentation of the arterial vessel types. Pressure segmentation of the vasculature has been shown by Davis et al. in U.S. Pat. No. 6,749,567 to be a valuable method of determining the pressure—volume segmentation of the vascular bed for determination of the volume distribution and vessel compliance for different types of vessels in the vascular system.

It is possible to implement this invention with multiple pressure applicators and pressure or volume sensors mounted adjacently along the path of arterial flow in the limb of the living subject. However, one preferred embodiment of the invention uses a combination of two bioimpedance based volume sensors for pulse volume determiniations in combination with a common blood pressure cuff for pressure application.

Two technology areas can be useful in the practice of the present invention: electrical modeling of the human body and pulse wave propagation models of the human circulatory system. The first postulates a model of the human body in terms of its electrical behavior, and relates physiological volume changes to changes in that electrical behavior. The second describes pressure wave propagation in the arteries due to blood volume changes.

Electrical Models of the Human Body

The “Parallel Conductor Theory” describes the human body as composed of various conductive elements that represent different materials of the human body composition.

The electrical impedance, Z. of a body component (bone, fat, muscle, blood, etc.) is a complex number, Z=R+jX, characterized by its resistance R and reactance X. The admittance, Y, of this component the inverse of the impedance, or 1/Z . The impedance, Z, of a column of material with a cross sectional area A (cm²) and length l (cm) is:

Acs (Impedance form) {1}

$$\begin{matrix}Z = \frac{{ρ}_{r} * l}{{A}_{cs}} ( Impedance form ) & \{ 1 \} \\ Y = \frac{{A}_{cs}}{{ρ}_{r} * l} ( Admittance form ) & \{ 2 \}\end{matrix}$$

where ρᵣ is the bulk resistivity of the measured material in ohm-cm.

In the parallel conductor hypothesis, a limb, or body segment, is equated to a series of parallel conductors in which the volume of blood in the limb is the only variable. The impedance of the whole limb is the sum of the several parallel impedances, Zᵢ, of its components, calculated according to the formula:
1/Z=1/Z₁+1/Z₂+1/Z₃ . . . +1/Zₙ
This relation is more easily expressed in terms of admittances of the limb components:
Y=Y₁+Y₂+Y₃ . . . +Yₙ

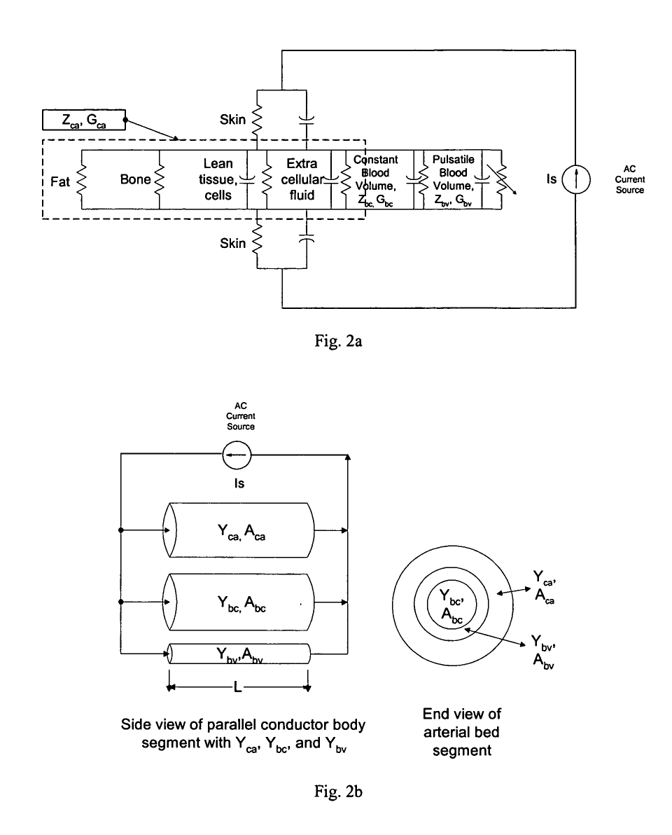

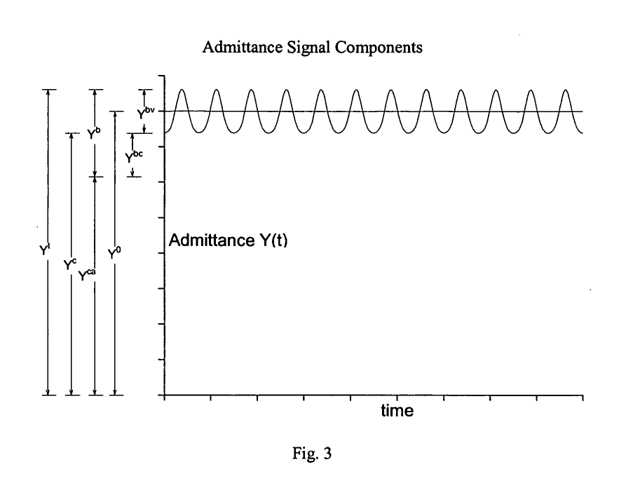

The parallel conductor model (FIGS. 2a, 2b and 3) is composed of three basic admittance values: Y_{ca}, Y_{bc} and Y_{bv}. Y_{ca} represents all non-blood elements in the body segment that are unchanging over the span of a cardiac cycle, Y_{bc} represents the basal, or constant, blood admittance in the segment over the cardiac cycle, and Y_{bv} represents the variable blood admittance. Y_{c} represents the sum of Y_{ca} and Y_{bc} and is the total portion of the segment admittance that does not vary; Y_{b} is the combination of Y_{bc} and Y_{bv}, and represents the total blood admittance; and Yₜ is the sum of all three basic admittances and is the total measured segmental admittance. The parallel conductor model can be expressed to show the total admittance of a body segment as;
1/Zₜ=1/Z_{bv}+1/Z_{c} (Impedance form) {3}
Yₜ=Y_{bv}+Y_{c} (Admittance form) {4}

It has been shown by Nyboer, Jan, Electrical Impedance Plethysmography, 2^{nd} Edition, Thomas Books, Springfield, Ill., 1970, that the arterial blood volume is proportional to the electrical conductance (1/R) in a section of the human body. This proportionality is dependent on the relationship between the volumetric changes occurring in the vascular bed due to the “pressure wave” caused by the heartbeat and the conductance or impedance value of the vasculature versus time. The ability to accurately and non-invasively measure volumetric changes in the vascular bed by impedance or conductance has been researched and discussed in the literature, e.g., Geddes, L A; and Sadler, C., “The Specific Resistance Of Blood At Body Temperature, Med. Biol. Eng. 11(3):336-339, 1973. Shankar, T. M. Ravi; Webster, John G.; and Shao, Shu-Yong; The Contribution of Vessel Volume Change and Blood Resistivity Change to the Electrical Impedance Pulse, IEEE Transactions on Biomedical Engineering, Vol. BME-32, No. 3, March 1985. Handbook of Biological Data, The National Academy of Sciences, National Research Council, Spector, W. S., Ed., W B Saunders, 1956. H. Shimazu, K. Yamakoshi, T. Togawa, M. Fukuoka, Evaluation of Parallel Conductor Theory for Measuring Human Limb Blood Flow by Electrical Admittance Plethysmography, January 1981, IEEE Transactions on Biomedical Engineering. Encyclopedia of Medical Devices and Instrumentation, John G. Webster, Editor in Chief, Volume 3, pg 1633, 1988, John Wiley & Sons, New York. Nyboer, Jan, Electrical Impedance Plethysmography, supra. Lifshitz, K. Electrical Impedance Cephalography, Electrode Guarding And Analog Study, Ann. N.Y. Acad. Sci. 170:532-549, 1970.

As shown below, all of these approaches require knowledge of the resistivity of the blood. Nyboer, J., “Electrical Impedance Plethysmography: A Physical And Physiologic Approach To Peripheral Vascular Study, Circulation, 2:811-821, 1950, applied the formula for the resistance of a homogeneous volume conductor to predict the relationship between impedance changes and blood volume changes. The electrical impedance Zₜ of a cylindrical body segment, such as a limb, can be expressed in terms of its cross sectional area A_{cs}, voltage electrode separation L, and tissue resistivity ρᵣₜ

$${Z}_{t} = \frac{{ρ}_{rt} * L}{{A}_{cs}} \qquad \{ 5 \}$$

Since the volume of a body segment, Vₜ=L A_{cs}, electrical resistance can be expressed in terms of the segmental volume,

$$\begin{matrix}{Z}_{t} = \frac{{ρ}_{rt} * {L}^{2}}{{V}_{t}} & \{ 6 \} \\ {V}_{t} = \frac{{ρ}_{rt} * {L}^{2}}{{Z}_{t}} & \{ 6 a \}\end{matrix}$$

Nyboer further assumed that the segmental blood volume change ΔV_{bv} could be modeled as the resistance change ΔZₜ in the segment due to change in blood volume electrically in parallel with the basal, or constant, tissue impedance Zₜ. This led to the well-known Nyboer Formula,

$$Δ {V}_{bv} = - {ρ}_{rb} * {L}^{2} * \frac{Δ {Z}_{t}}{{Z}_{t}^{2}} \qquad \{ 7 \}$$

where ρ_{rb} is the resistivity of blood.
Vessel Wall Tension and Compliance

It can be seen from FIG. 1b that the Transmural Pressure (Pₜₘ) of the vessel is determined by the difference between the internal pressure and the external pressure of the vessel. The external pressure (P_{c}) which normally works against the outside of the vessel is atmospheric pressure plus some force produced by tissues. The internal fluid pressure (Pₒ) of the vessel is produced by the fluid volume in the vessel plus hydrostatic pressure produced by the column of fluid above and below the region of interest.
Pₜₘ=Pₒ−P_{c}. {8}

Compliance of the vessel is generally defined as ΔV/ΔP and herein the wall tension of the vessel is defined as the inversion of Compliance (1/C). It can be seen from FIG. 1b and Equation {8} that when Pₒ=P_{c}, then Pₜₘ=0. This relationship is the physical reason for the maximum oscillation of the blood pressure cuff commonly referred to in automated oscillometric blood pressure monitoring. (References for Oscillometric Blood Pressure Measurement is made to “Principles of Applied Biomedical Instrumentation”, 3^{rd} Ed, L. A. Geddes, L. E. Baker, John Wiley and Sons, 1989 and Mauck G W, Smith C R, Geddes L A, Bourland J D. The meaning of the point of maximum oscillations in cuff pressure in the indirect measurement of blood pressure-part ii. J Biomech Eng. 1980; 102: 28-33.) When the vessel wall is, in effect, unloaded, the pulse pressure wave can maximally change the volume of the vessel and therefore maximally impinge the largest pressure change into the constraining blood pressure cuff. It is the relaxation or unloading of the vessel wall that allows for the maximal wall motion during each cardiac cycle, and therefore the maximum pressure oscillation in the cuff. This demonstrates how externally applied pressure can change the compliance of the vessel wall. Similarly, any means by which Pₒ can be changed will have the inverse but similar affect on the vessel wall attributes as is observed by changing P_{c}. Any means for changing hydrostatic pressure within the vessel will have a similar but inverse impact on the vessel wall compliance by changing the Pₒ pressure of FIG. 1b.

Pulse Wave Propagation Models

The Moens-Korteweg equation, first published around 1878, is the most cited work dealing with pressure wave velocity in an artery. It is given by:

$$v = \sqrt{\frac{E * h}{2 * {ρ}_{b} * {r}_{i}}} \qquad \{ 9 \}$$

where v=pulse wave velocity, E=elastic modulus of the vessel wall, h=vessel wall thickness, ρ_{b}=density of the blood, and rᵢ=the vessel inside radius. The pulse wave velocity is the speed at which the pressure pulse propagates along the vessel.

Bramwell J. C. and Hill A. V., The Velocity Of Pulse Wave In Man, Proc. Soc. Exp. Biol. Med., 1922; 93: 298-306, modified the Moens-Korteweg equation to the form:

$$v = \sqrt{\frac{{V}_{b}}{{ρ}_{b}} * \frac{Δ P}{Δ {V}_{b}}} \qquad \{ 10 a \}$$

where v=pulse velocity, V_{b}=basal volume of the blood in a vessel, ΔP=transmural pressure change due to the pulse, ρ_{b}=blood density, and ΔV_{b}=volume change of the blood in the vessel due to the pulse. Here the velocity of the pulse wave is expressed in terms of volume, pressure, and density. Prior investigations into vascular behavior, which use the Moens-Korteweg Equation {9} for vascular modeling, have chosen to insert a nominal or invasively determined value for ρ_{b} in the use of this equation.

It will be noted that in this document the symbol ρ is used to refer to both the density of a substance in gm/cm³ and to the resistivity of a substance in Ω*cm. This overlapping of symbols is unfortunate, but dictated by convention. In this document resistivity will always have a first subscript of r, as in ρ_{rb} for the resistivity of blood. Whenever ρ does not have a first subscript of r it refers to density, as in ρ_{b} for the density of blood.

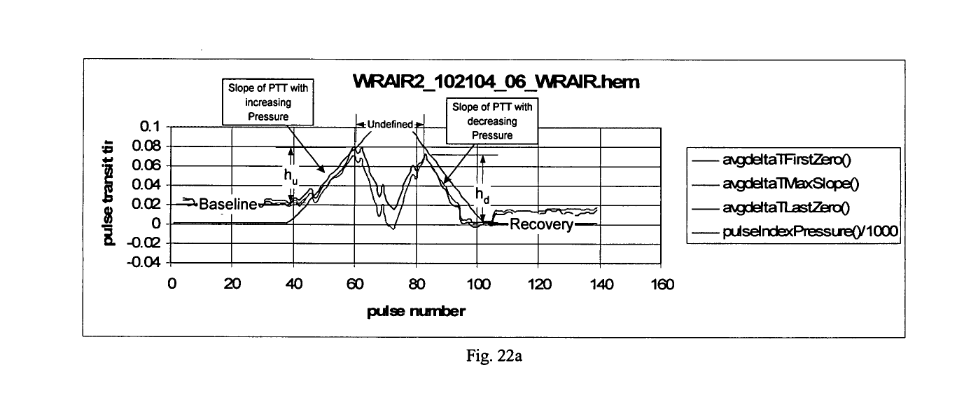

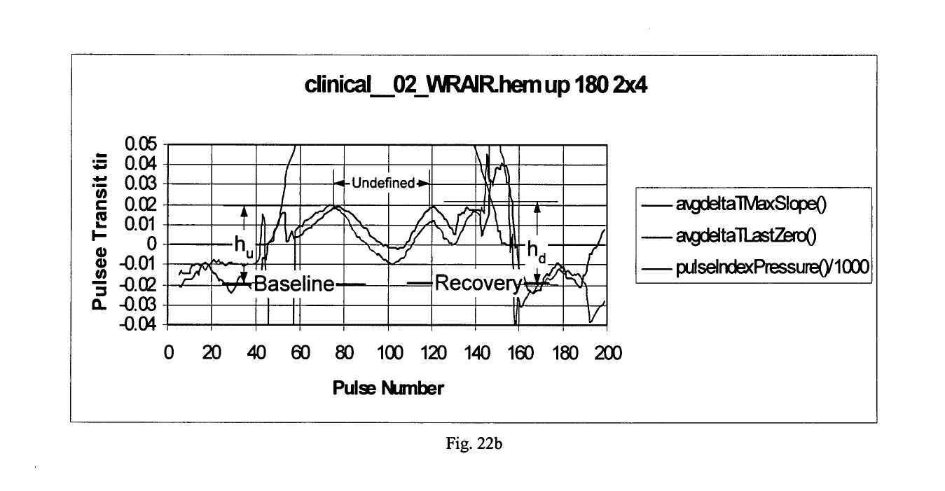

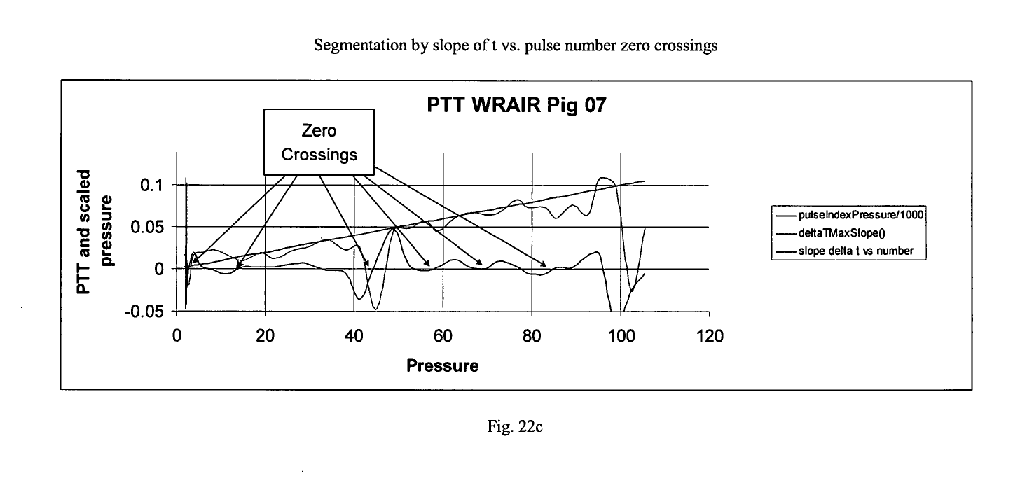

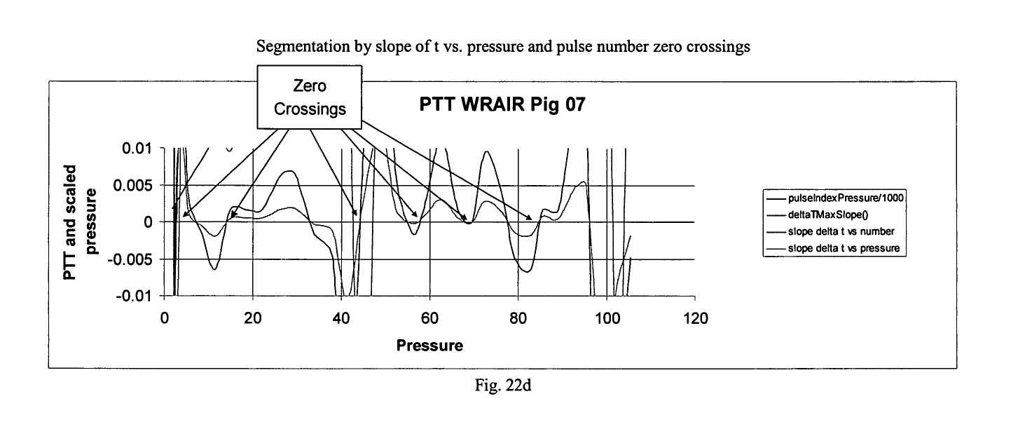

The Bramwell-Hill equation can be rearranged in equation 10b to show the relationship between the compliance of the arteries as defined as the rate of change in volume due to a change in pressure as a function of the volume of fluid, the density of fluid, and the velocity of the pulse wave in the vessel. Equation 10b shows that the compliance and the velocity of the pulse wave have an inverse relationship, therefore confirming that as the compliance increases, then the velocity of the pulse wave would decrease and the pulse transit time would increase as demonstrated by the experimental data shown in FIGS. 22a and 22b.

$$\frac{Δ {V}_{b}}{Δ P} = \frac{{V}_{b}}{{ρ}_{b} {v}^{2}} \qquad \{ 10 b \}$$

Since we have defined the velocity of the pulse wave v=L/t then the relationship shown in equation 10c demonstrates the linear relationship between the pulse transit time (t) and the compliance of the vessel.

$$\begin{matrix}\frac{Δ {V}_{b}}{Δ P} = \frac{{V}_{b}}{{ρ}_{b}} * \frac{{t}^{2}}{{L}^{2}} & \{ 10 c \} \\ ( \frac{Δ {V}_{b}}{Δ P ( {V}_{b} )} ) ( {ρ}_{b} {L}^{2} ) = {t}^{2} & \{ 10 d \} \\ \sqrt{( C * \frac{1}{( {V}_{b} )} ) ( {ρ}_{b} {L}^{2} ) = t} & \{ 10 e \}\end{matrix}$$

Since ρ_{b} and L are constants, the only variables in equation 10c that can be affecting the change in t are P and V in equation 10d. In equation 10e, the ΔV_{b}/ΔP compliance term has been replaced by a single term C for compliance. This compliance is called the dynamic compliance of the vessel because it only relates to the time varying elements of pressure and volume in the vessel, and does not incorporate the effects of the static pressure and volume in the vessel.

Since the Bramwell-Hill equation describes the behavior of a pulse wave through an otherwise static system of vessels, what may not be readily apparent from the Bramwell-Hill equation is the affect of changes to the static condition of the transmural pressure from one pulse wave to the next. Equations 10a, 10b, and 10c assume a baseline or static state of compliance and model the dynamic compliance behaviors of the vessel. Since the vessel has higher pressure on the inside than the outside, the static transmural pressure vector is from inside the vessel to outside the vessel. Each new pulse wave that propagates through the region of vessels experiences a static compliance to which it contributes additional volume and pressure to the system. This static compliance we will call the “Baseline Compliance” for the system of vessels in our region of interest. In a vascular system this baseline compliance would be defined by the mean pressure of the vessel and the volume of fluid in the vessel. In this static compliance model the ΔV_{b} term would equal the V_{b} term and they would cancel each other out. The ΔP term would be equal to the mean pressure of the vessel. We can see from this analysis in equation 10f, that the static or baseline pulse transit time is a function of the mean arterial pressure and the density of the fluid.

$$t = L \sqrt{\frac{{ρ}_{b}}{Δ P}} ( for baseline pulse transit time ) \qquad \{ 10 f \}$$

Equation 10d shows that by either increasing external pressure or reducing the internal static pressure of the vessel, the transmural pressure ΔP will be reduced. By reducing ΔP, t must increase.

$$t = L \sqrt{\frac{{ρ}_{b}}{Δ P} * \frac{Δ {V}_{b}}{{V}_{b}}} ( for all pulse transit time ) \qquad \{ 10 g \}$$

The Bramwell-Hill equation models the dynamic behavior of a vessel which is statically established with residual fluid volume V_{b} and mean pressure Pₘ. If ΔP is defined as the transmural pressure of the vessel and Pₛₜ as the static pressure of the vessel relative to the external pressure P_{c} (FIG. 1b), then ΔP═Pₛₜ+dP at every point in time that the vessel is free standing with no manipulation of either the internal static pressure (Pₒ, FIG. 1b) or the external environmental pressure (P_{c}, FIG. 1b). However, manipulating either the internal static pressure or the external environmental pressure, changes Pₛₜ, and the next pulse wave dP will produce a different t due to the change of the static state of the vessel as shown in equation 10h. It is easy to interpret ΔP in this relationship as representing only the time varying transmural pressure dP while ignoring the static transmural pressure Pₛₜ=Pₒ−P_{c}. In one embodiment, this invention uses methods for changing Pₛₜ and measuring t for a sequence of values for Pₛₜ and relating the behavior of t to values of Pₛₜ.

$$t = L \sqrt{\frac{{ρ}_{b}}{( {P}_{st} \div dP )} * \frac{Δ {V}_{b}}{{V}_{b}}} ( for all pulse transit time ) \qquad \{ 10 h \}$$

The prior discussion of the Moens-Korteweg Equation {9} and Bramwell-Hill Equation describing the propagation velocity of the pulse wave in arteries (or any other elastic wall vessel), illustrates the co-dependent relationship that exists between the propagation velocity (v) of the pulse wave in such a medium and the pressure change (ΔP) across the vessel wall. Furthermore, it is seen in {10b} that the compliance of the vessel, defined as ΔV/ΔP, has a dependent relationship with the velocity of the wave, the residual fluid volume of the vessel, and the density of the fluid.

Therefore, the velocity of the pulse wave is a function of the compliance of the vessel wall, the residual fluid volume of the vessel, and the density of the fluid. Any modification of the static system state affecting the transmural pressure, the volume of the vessel, or the density of the fluid affects the pulse velocity through that region. This demonstrates a mechanism for determining the compliance attributes of the vessels by use of measurements of the pulse velocity or pulse transit time through the region of interest. The discontinuities that occur in the behavior of Pulse Transit Time and Pulse Wave Velocity versus pressure through the region are indicative of the state transition boundaries as shown in FIG. 1a.

Calculating Vessel Wall Compliance from Pulse Transit Time or Pulse Wave Velocity.

The total compliance of the vessel for any static state of hydration volume and pressure is shown in equation 10i.

$$\frac{t}{\sqrt{{ρ}_{b}} L} = \sqrt{\frac{Δ {V}_{b}}{( {P}_{st} \div dP ) {V}_{b}}} \\ ( for all pulse transit times / static compliances ) \qquad \{ 10 i \}$$

The right side of the equation is the total compliance for the vessel for any state of hydration volume. From this we can see that the pulse transition time (t) is the lone variable for determining the total compliance attribute of the vessel, and the density of the fluid (ρ_{b}) and the path length (L) are considered constants over the period of measurements to be made in the region of vessels. Equation 10i is the inverse of the velocity relationship to compliance as the ratio of t/L=1/v.
Determining V_{b} for the Region of Vessels

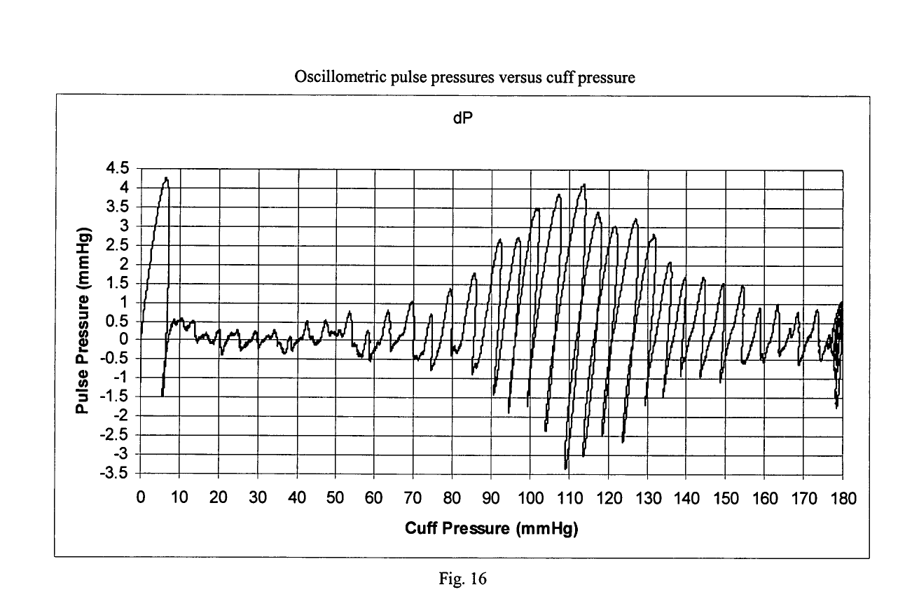

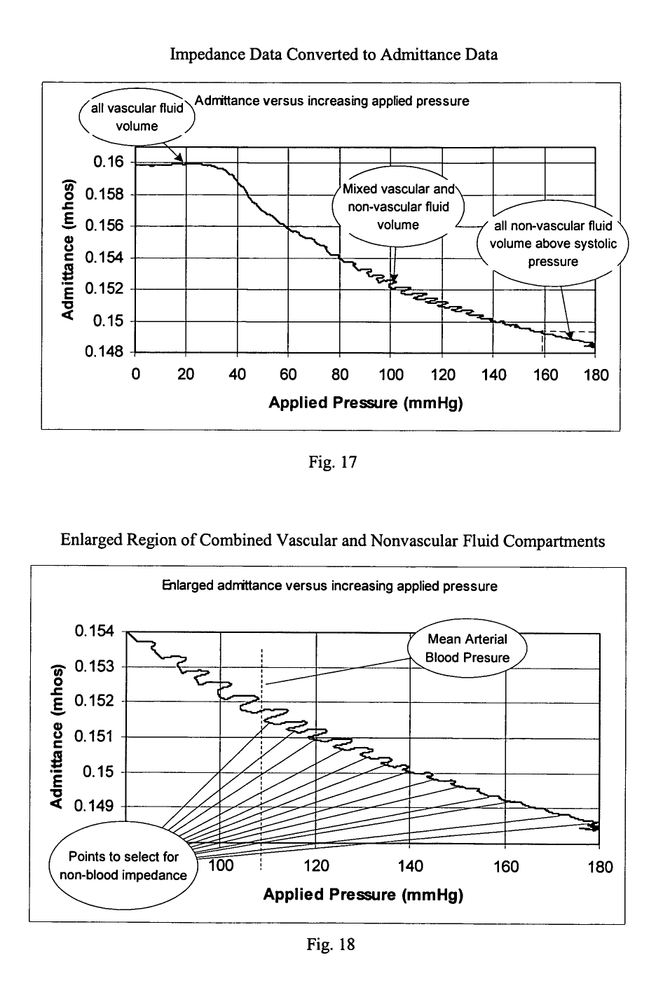

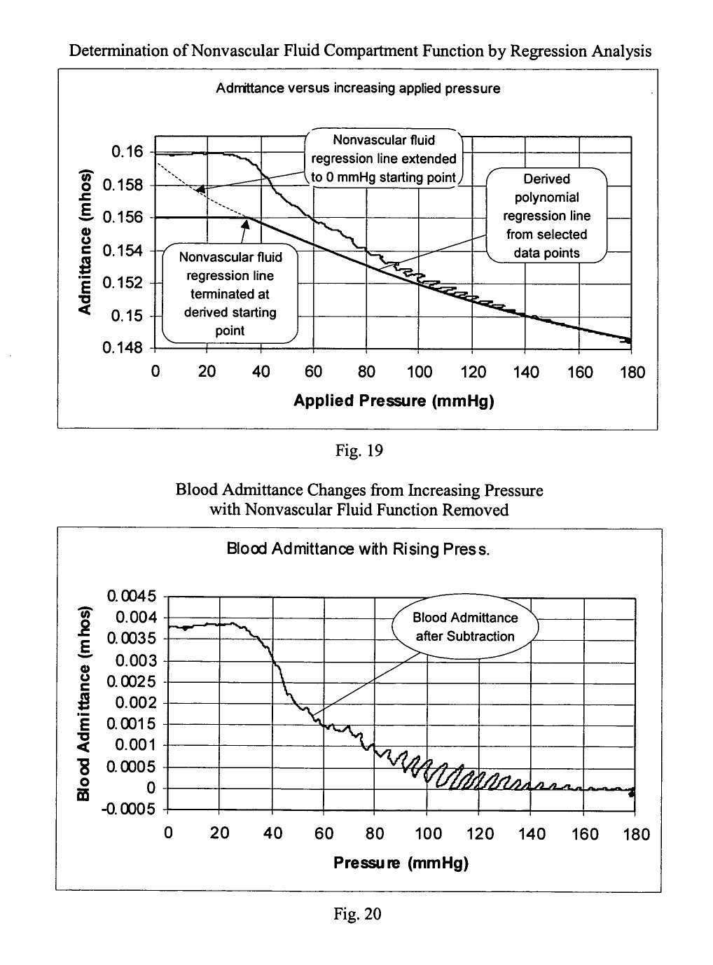

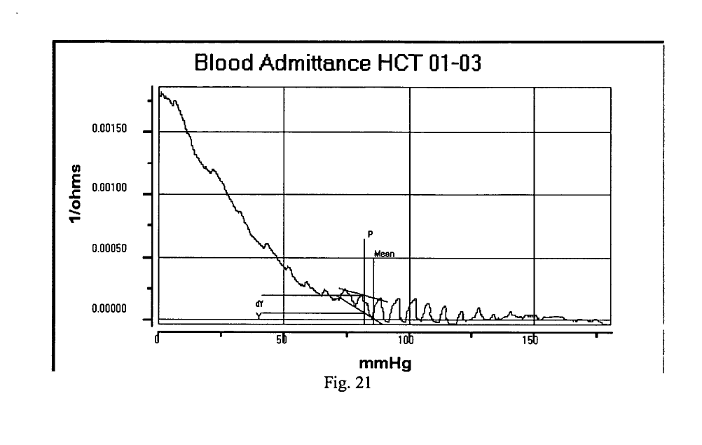

V_{b} is the static fluid volume in the region of vessels. This value is difficult to determine noninvasively since it is static and unchanging for any given transmural pressure. However, the methods described herein allow for calculating this value from a series of measurements. First the mean pressure of the vessel in equation 10i must be determined in order to be able to calculate Pₛₜ=Pₘ−P_{c}. Pₘ and the pulse pressure dP may be determined for the vessel by conventional oscillometric methods that are well known in the literature and prior art (FIG. 16). Once Pₘ is known, Pₛₜ can be calculated for each P_{c} pressure that is applied to the vessel. ΔV_{b} can be determined by measuring the height of the pulse volume as shown in FIG. 1d for each pulse wave that passes through the region of vessels. ρ_{b} and L are known constants. V_{b} can be directly determined once these parameters are known from the measurement at each pressure.

Determining Segmentation of the Vasculature from the Pulse Transit Time versus Pressure

In one embodiment, segmentation of the vasculature can be accomplished with this invention, such as demonstrated in FIGS. 22c and 22d. In these examples the pulse transit time is plotted versus applied pressure. In FIG. 22c, the slopes of three sequential pulse transit times against the pulse numbers are plotted versus applied pressure demonstrating a method of segmentation of the vasculature by identifying the zero crossings of the oscillating slope function of the pulse transit times. The same process can be shown using pulse wave velocity. FIG. 22d shows the same data with the slope of the pulse transit times calculated using the applied pressure values for each pulse transit time demonstrating that the linear pressure values produce the same resulting zero crossing functions. As will be appreciated by those skilled in the art following the teachings herein provided, other alternative mathematical methods may be used to determine segmentation of the vasculature using the pulse transit time and the pulse wave velocity values versus pressure and are included herein.

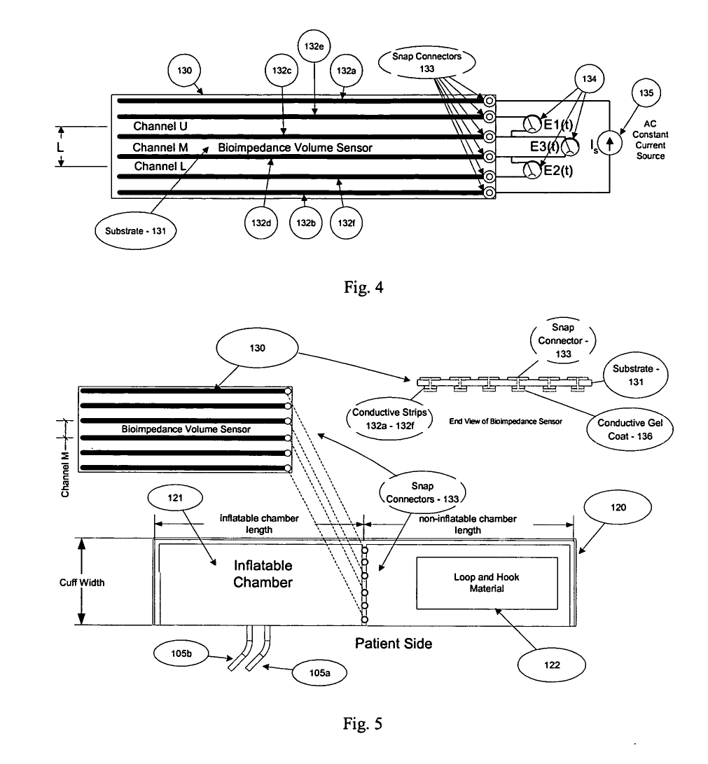

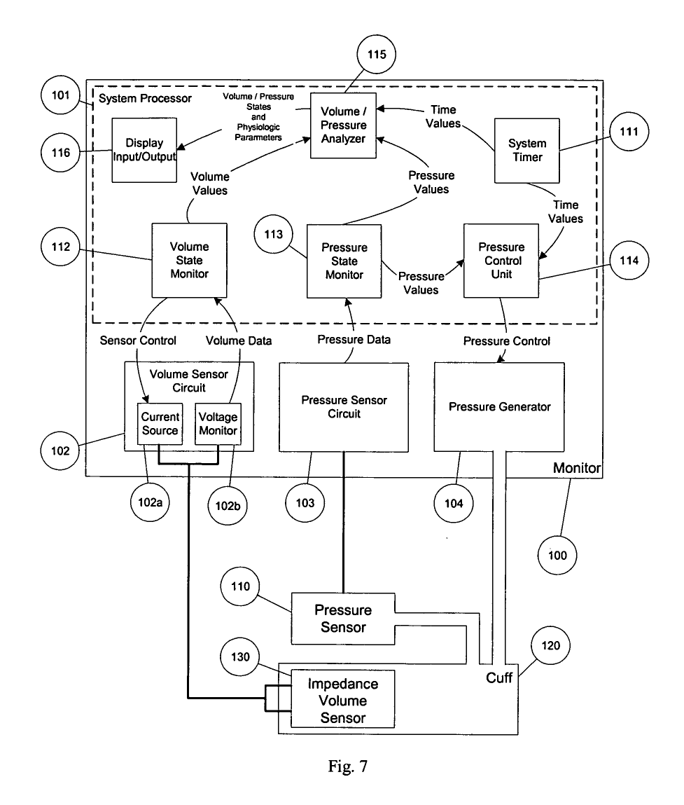

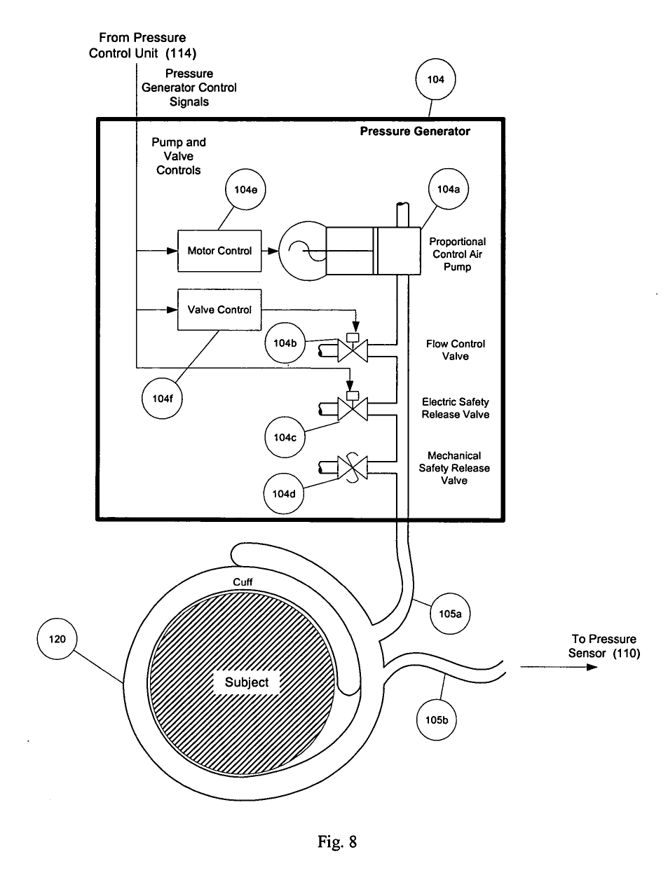

In one embodiment of this invention, as shown in FIGS. 4-13, and summarized in the block diagram of FIG. 7, the method is performed using an impedance volume sensor 130, an inflatable cuff pressure generator 120, a pressure sensor 1110, and a Monitor 100.

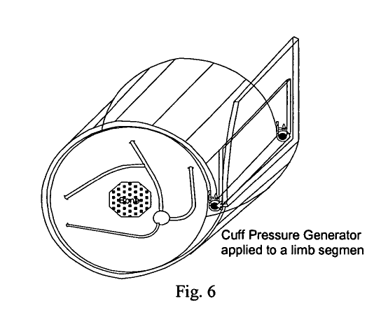

Referring to FIGS. 4 and 5, the impedance volume sensor 130 may be a bio-impedance sensor comprised of a matrix of four or more parallel conductive lines, here 132a-132f, fixed to a flexible substrate material 131, e.g., such as or similar to MYLAR®, with snap connectors 133 on one end of each conductive line as shown in FIGS. 4 and 5. It is desirable that the substrate firmly maintains the separation between conductive lines, as further discussed below. The impedance volume sensor 130 is fitted to the patient side of the inflatable-cuff pressure generator 120, i.e., the side intended to be applied to the surface of the body of the subject. The alignment of pressure generator 120 and impedance volume sensor 130 is such that the volume sensor is centered under the inflatable bladder portion of the inflatable cuff pressure generator 120.

The impedance volume sensor shown in FIGS. 4 and 5 derives its measurements from conductive lines 132a-132f that may be produced with conductive paint or other material suitable for bio-impedance monitoring. Furthermore, the conductive lines may be coated with a gel material 136 suitable for reducing the high resistance layer of the skin of the subject, without causing adverse chemical reaction. Alternatively, point electrodes might be used in the impedance sensor, although signal to noise issues may result.

The excitation leads 132a and 132b are the input and output connections for the AC constant amplitude current source 135. The constant current source delivers a nominal 50 kHz, 1.2 mA RMS, constant amplitude, alternating current to the body region of the subject. It is anticipated by the inventors that the constant amplitude current is desirably an alternating current of a frequency capable of producing a uniform current density within the body region for normal operation of the invention. This constant amplitude alternating current establishes a circuit through the patient limb creating a voltage drop along the current path that is proportional to the impedance of the tissue bed. Voltage drops are measured between electrode elements that generate voltages E1, E2, and E3 134 as shown in FIG. 4.

The distance between the center conductive lines 132c and 132d of the impedance volume sensor 130 defines the width of the middle measurement channel (Channel M), and therefore defines the body region that will be used by the invention to measure blood volumes. A desirable separation of the conductive lines, which define Channel M of impedance volume sensor 130, is less than the cuff width divided by five. Furthermore, Channel M should be located in the middle of the inflatable cuff width. Impedance volume sensor 130 is preferably coextensive with but not wider than the cuff 120. The sensing leads 132c, 132d, 132e, and 132f should be positioned between the excitation leads 132a and 132b.

In FIG. 4, the upper and lower channels (Channels U and L) formed by the upper two sensing leads 132c and 132e and the lower two sensing leads 132d and 132f are used for the pulse wave velocity and pulse transit time measurements. Thus the separation between these two channels represents the length L over which the pulse wave velocity and pulse transit time is measured. This dimension is constant for all measurements using the same geometry sensor and therefore can be treated as a calibration constant in the pulse wave velocity and pulse transit time calculations:

The impedance volume sensor 130 is attached to the inflatable cuff 120 by a connector system. This could be, for example, individual snap connectors 133, or a connector bank with some latching mechanism for locking the impedance volume sensor into place and creating the electrical circuits for the impedance volume sensing.

Once the impedance volume sensor is mated with the inflatable cuff, the combination unit 120 and 130 may be applied circumferentially to a limb of the subject 99 as shown in FIG. 6. The inflatable cuff is preferably wrapped around the upper arm of the subject with the impedance volume sensor applied directly to the skin of the subject. The inflatable cuff is wrapped snugly around the limb of the subject with the conductive lines preferably running at substantially a right angle to the length of the limb. Adhesive may be used to secure the conductive lines to the subject.

Pressure applicator or generator 120 is desirably an inflatable cuff, as shown in FIG. 5, using air or other fluid for inflation of the cuff bladder or chamber 121. The cuff may be circumferentially fitted around an appendage of the subject (FIG. 6) including, but not limited to, an arm, leg, finger, or toe in such a manner as to be capable of generating pressure against the body region of the subject. The pressure generator 120 is secured to the subject in this exemplary embodiment by hook and loop material 122 which is commonly used for blood pressure cuff application.

A common blood pressure cuff, as shown in FIG. 5, is the prevalent method available for applying pressure against a body region for physiologic parameter measurement. As it may be commercially advantageous to use commonly available blood pressure cuffs for pressure generation in certain aspects of the present invention, the inventors have observed that a reasonably accurate determination of blood admittance, or volume, versus pressure data can be accomplished if the volume measuring region defined by the width between sensor leads 132c, 132d of the bio-impedance sensor 130 (FIG. 4), is kept narrow relative to the width of the inflatable bladder 121. It is also desirable that if the volume measuring region defined by sensors 132c, 132d is located at the center of the inflatable bladder as shown in FIG. 4. Desirably, the width of this region should not exceed one fifth of the cuff width for reasonably accurate determinations of volume/pressure values.

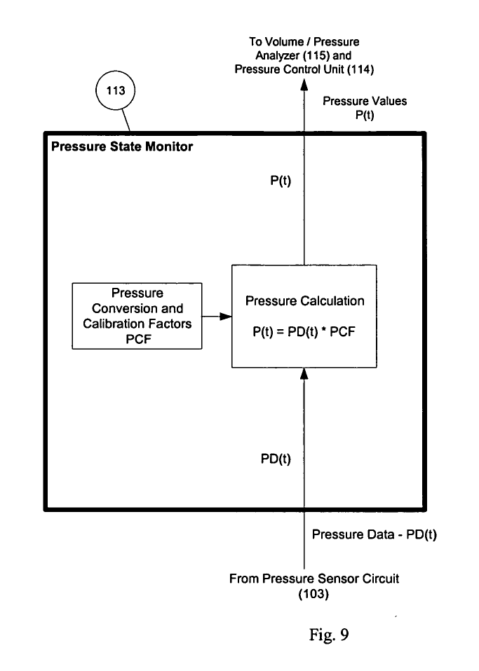

The pressure sensor 110 shown in FIG. 7, preferably measures the pressure produced at the cuff 120, rather than at the pump for greater accuracy. The pressure sensor 110 produces an electronic signal representing pressure data. The pressure signal is received by the pressure sensor circuit 103 which produces pressure data. The pressure data is in turn received by the pressure state monitor 113 and processed into pressure values as shown in FIG. 9. The pressure values are sent to the pressure control unit 114 for feedback control and to the volume pressure analyzer 115 for analysis.

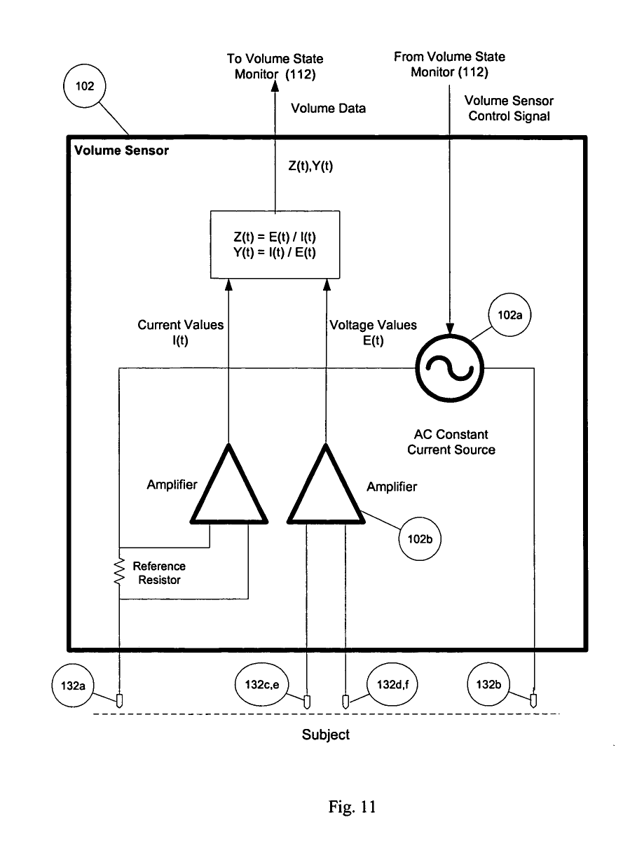

As shown in the block diagram of FIG. 7, the monitor 100 comprises four major subsections: the system processor and calculator 101, the volume sensor circuit 102, the pressure sensor circuit 103, and the pressure generator 104. In this embodiment the volume sensor circuit 102 (FIG. 11), the pressure sensor circuit 103, and the pressure generator 104 (FIG. 8) are seen to be implemented in hardware. Each of these functions could be accomplished in many different ways, and only a representative solution is presented. In this implementation the system processor 101 is implemented as a microprocessor and the various functions 111 through 116 illustrated in FIG. 7 and their necessary calculations are performed in software. They could, however, be performed by dedicated hardware.

The volume sensor circuit 102, as seen in FIG. 11, consists of an AC constant current source 102a and an amplifier for each channel 102b to measure the voltage generated across the channel electrodes by the applied current from the current source 102a. The volume sensor circuit 102 produces volume data including any data indicative of volume or volume changes in the body region of the subject. Therefore, the volume sensor circuit 102 may measure absolute, calibrated, relative or proportional (admittance) volume data from the body region. In addition, there is a reference channel for correcting for any inaccuracies in the constant current source. An analog to digital conversion is then performed on the output of these amplifiers and the results combined as shown in FIG. 11 to produce admittance volume data. The admittance volume data is passed to the system processor.

The pressure sensor circuit 103 amplifies and converts the analog electrical signal output from the pressure sensor to digital data. This digital data is then passed on to the system processor 101 as pressure data. In addition the pressure sensor circuit provides whatever source or stimulation is required by the particular type of pressure sensor used.

The pressure generator 104, as seen in FIG. 8, consists of a pump 104a and several valves 104b, 104c, and 104d along with control circuitry 104e and 104f to control them. The pressure generator 104 may include any means and method for applying and relieving pressure to a body region of a subject in a controlled manner. The pressure generator 104 may be capable of applying increasing or decreasing pressure at a linear, nonlinear, or step-wise rate of change of pressure versus time. Furthermore, the pressure generator 104 can be capable of holding pressure at a pressure level for a period of time, or dithering above and below a pressure level over a period of time. While more elaborate configurations employing multiple pumps and more valves can be used to improve the linearity, accuracy, and smoothness of the pressure generation, the configuration shown in FIG. 8 is sufficient to demonstrate the basic function required. These more elaborate configurations are considered to be possible in other embodiments of the invention.

The system processor 101 shown in FIG. 7 shows several functional blocks that would be implemented in software in this embodiment. These are the system timer 111, the volume state monitor 112, the pressure state monitor 113, the pressure control unit 114, the volume/pressure analyzer 115, and the display input/output 116. These blocks represent steps in the analysis of the data from raw A-to-D converter data to finished physiologic parameters.

The system timer 111 is a highly accurate time base for all of the functions of the system processor. It provides the sample rate timing as well as timing for controlling the various functions of the monitor 100. In this embodiment of the invention the time base is derived from the system clock of the microprocessor.

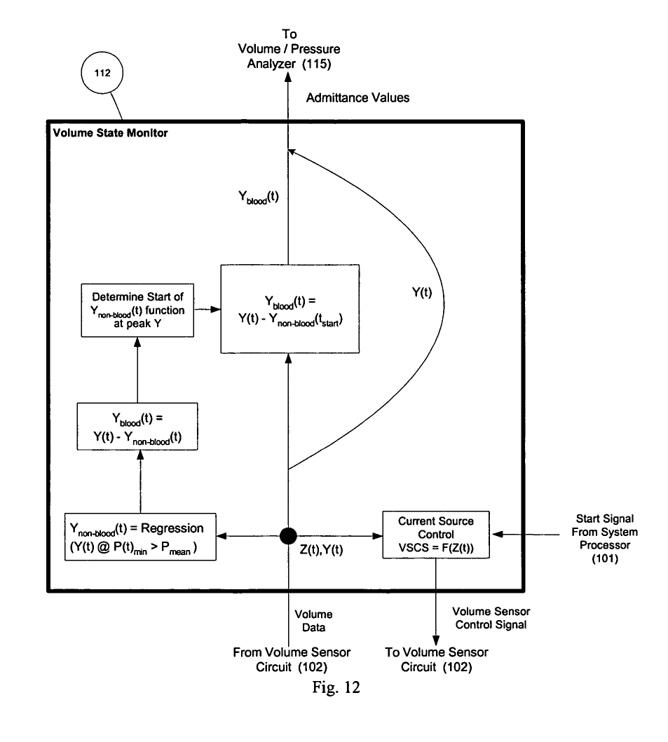

The volume state monitor 112 (FIG. 12) is a computational routine that performs the conversion of raw volume data from the volume sensor circuit 102 in the form of impedances or admittances, to actual blood volumes. An array is made of this data as a function of time.

The pressure state monitor 113 (FIG. 9) is a computational routine that performs the conversion of raw pressure data to calibrated pressure.

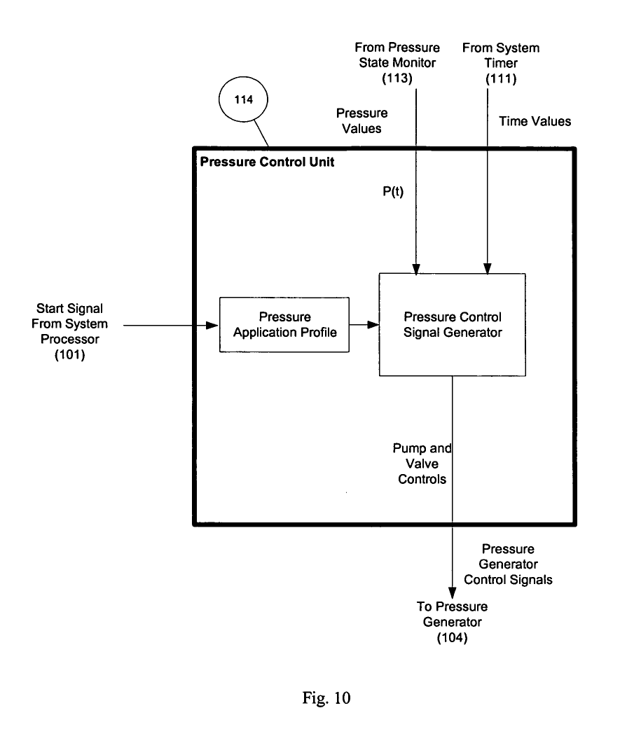

The pressure control unit 114 (FIG. 10) is a control routine which uses the pressure values from the pressure state monitor 113 together with a programmed pressure profile to control the pressure generator 104. It operates in such a manner as to cause the pressure in the cuff to precisely track the pressure profile up or down over time.

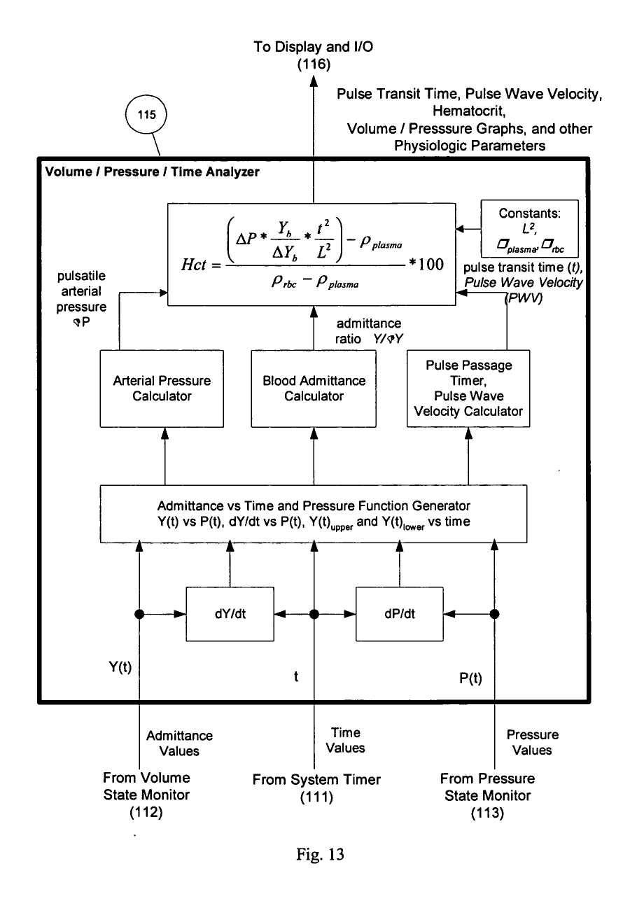

The volume/pressure analyzer 115 (FIG. 13) performs the fundamental calculations resulting in output parameters. In the case of this embodiment, it uses the pressure and admittance values supplied by the volume state monitor 112 and the pressure state monitor 113 together with timing supplied by the system timer 111 to solve for the pulse wave velocity and pulse transit time.

The display input/output 116 is the source for user input to the monitor and is the output device for the physiologic parameters which are the results of the above calculations. It is anticipated that many other parameters and system variables could be displayed on the device.

In one embodiment of this invention, monitor 100 begins a measurement cycle when the system processor 101 generates a “start” signal. The pressure control unit 114 generates pump and valve signals for the pressure generator 104 (FIG. 8), activating the air pump 104a and closing the control valve 104b. The electrical and mechanical safety valves 104c and 104d are normally closed except in the case of a mechanical or electrical fault exceeding allowed limits for safe operation. Pressure generator 104 inflates the cuff 120 according to a pressure application profile in pressure control unit 114. The pressure application profile is a prescribed inflation/deflation rate and manner suitable for measuring pressure changes in the cuff by the pressure sensor 110 and volume changes by the impedance volume sensor 130.

The pressure signal from the pressure sensor 110 is amplified by the pressure sensor circuit 103 and applied to the pressure state monitor 113. The pressure state monitor 113 (see also FIG. 9) calibrates the pressure signal from the pressure sensor 110 for use by the pressure control unit 114 (see also FIG. 10) and the volume/pressure analyzer 115 (FIG. 13). The pressure control unit 114 produces control signals for the pressure generator 104 for controlling the rate and direction of pressure change against the body. The pressure control unit 114 also controls the limits of pressure that is applied to the body region of the subject. The pressure generator 104 may apply or relieve pressure against the body region.

In response to the “start” signal from 101, the volume state monitor 112 produces volume sensor control signals for the volume sensor circuit 102. The volume sensor control signal starts the current source 102a to concurrently apply a constant current to the subject through the forcing sensor electrodes 132a and 132b on the volume sensor 130. As the pressure increases and decreases on the body segment under the volume sensor 130 due to the action of the pressure generator 104 and cuff 120, a voltage is concurrently measured between the sensing electrodes of the Upper, Middle, and Lower channels 132c, 132d, 132e, and 132f on the volume sensor 130 by the voltage monitor 102b. The volume sensor circuit 102 converts the current and voltage signals into real-time impedance (Z(t)) and admittance (Y(t)) signals.

The volume state monitor 112 receives admittance data from the volume sensor circuit 102 and processes the body segment admittance data into blood admittance values using the non-blood subtraction method described below. The volume state monitor 112 also produces volume sensor control signals for stimulating and controlling the volume sensor circuit 102. Blood volume values may be absolute, calibrated, relative, or proportional i.e. admittance to actual volumes of the subject. The volume sensor circuit 102 and volume state monitor 112 may, in combination, perform volume value determinations using any method of noninvasive detection of volume or volume changes in a body region. This includes, but is not limited to, methods of bio-impedance as illustrated, ultrasound, optical absorption, optical diffusion, optical reflection, all forms of electromagnetic energy absorption, magnetic resonance, piezoelectric, tonometric, and mechanical displacement.

As further seen in FIGS. 9-13, the volume pressure analyzer 115 acquires admittance volume values from the volume state monitor 112 while the pressure generator is increasing pressure against the body region in a linear, non-linear, or step wise manner, or while the pressure generator is holding pressure constant against the body region, or while the pressure generator is decreasing pressure against the body region in a linear, nonlinear, or step-wise manner. The volume pressure analyzer 115 receives concurrent volume values and pressure values for analysis and presentation, as illustrated by the data flow arrows. The volume pressure analyzer 115 compares the upper and lower channel real-time volume data to calculate the passage time of the pulse due to a cardiac cycle. In addition it analyzes the volume values versus pressure values of the middle channel to determine arterial pressures and calculate pulse pressure. Finally the volume pressure analyzer 115 calculates the pulse wave velocity and pulse transit time measurement. These values are then plotted versus the applied external pressure or internal hydrostatic pressure and used to calculate the arterial wall tension and vascular segmentation of the subject.

The result of this calculation is presented to the operator by the display input/output 116 as the subject's pulse wave velocity (PWV), pulse transit time (PTT), changes in vascular wall tension and vascular filling pressures for vascular segmentation. By this time the cuff is fully deflated and the instrument is set for the next reading.

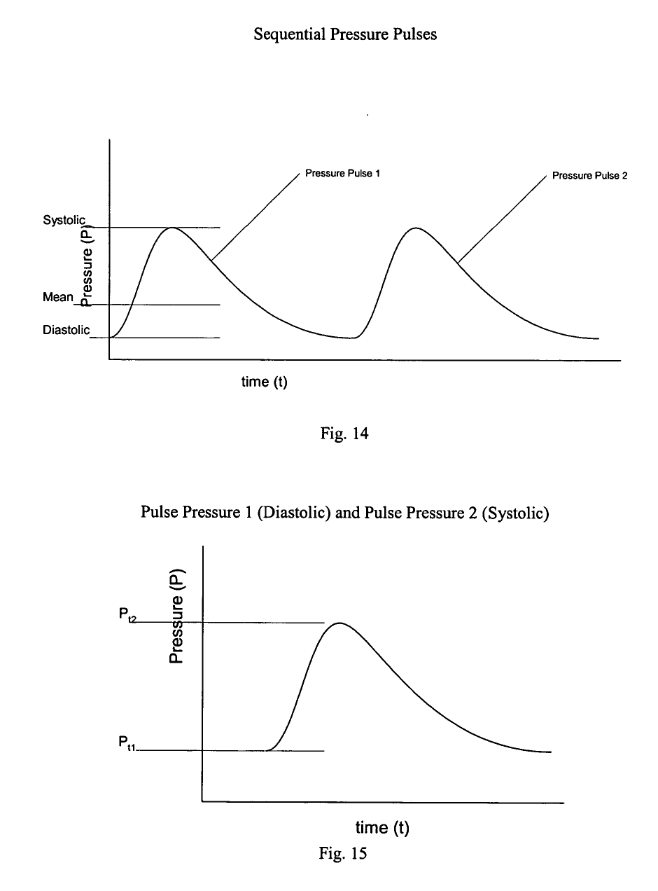

In one preferred embodiment, the voltages measured across the electrode pairs are proportional to the impedance of the tissue bed of the limb beneath the electrode pairs according to Ohm's Law (Z=E/I). The limb of a living subject is a non-homogenous material comprising lean muscle, blood, extravascular fluids, bone, and fat. The various impedance components of the limb are modeled as a parallel impedance model as shown in FIGS. 2a and 2b. The impedance of a limb segment of a living subject is comprised of fixed (unchanging) parts and variable (changing) parts as can be seen in FIG. 3. For a living subject the variable part of the impedance in a limb segment is the pulsatile volume of blood that propagates through the vascular system following every heart beat producing a propagating pressure wave (FIG. 14). The pressure pulse generated by each heart beat propagates through the vascular system at a velocity that is dependent on the volume of the vessel, the density of the blood, the peripheral vascular resistance, and the compliance of the vessel, which is defined as the change in volume generated by the pulse divided by the change in pressure. The lowest pressure that exists in the vessel at the end of each cardiac cycle is known as the Diastolic Pressure (FIGS. 14 and 15) and the highest pressure that is reached within the vessel is known as Systolic Pressure. The difference in these two pressures represents the pulse pressure propagating through the arteries. ΔP=P_{systolic}−P_{diastolic}. When relationships such as Equation {13} are rearranged, it is seen that the blood density, Pb, can be determined when the volume of the segment, the velocity of the pulse and the compliance of the vascular bed are known. In practice it can be difficult to determine the volumes of the vascular bed noninvasively. By using admittance as the sensing means, the volume parameters are replaced by a ratio of admittance values at the different pressure levels that define ΔP. This reduction of terms makes the method achievable and practical.

Arterial Pulse Pressure

In one embodiment of this invention, the arterial pulse pressure is obtained by determining the difference between the systolic pressure and the diastolic pressure. There are many well-known methods for determining arterial pressures non-invasively using a pneumatic cuff such as described above. The current embodiment of the invention uses the oscillometric method for blood pressure determination, but other techniques are possible.

Pulse Transit Time

In one embodiment of this invention, the pulse transit time is measured between the electrode pair of the Upper Channel 132c and 132e and the electrode pair of the Lower Channel 132d and 132f (FIG. 4), both of which are positioned under the sensor cuff. A variety of features of the wave-induced, time-varying admittance signal measured by these electrode pairs can be used to determine pulse transit time. These features include (FIG. 1d), but are not limited to, the peak of the wave (tₚ), the foot of the wave (t_{f}), the point of most rapid transition of the wave (tₘₛ), or the correlation time delay between a series of waves. For the preferred embodiment, tₘₛ has demonstrated the best reliability and repeatablity of the three timing methods.

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

What is claimed is:

1. A method of noninvasively obtaining a physiological parameter of a fluid vessel, the method comprising:
applying a series of pressure values to a region of the vessel to adjust the transmural pressure of the vessel wall;
measuring at each of the pressure values at least one of a pulse transit time and a pulse wave velocity through the region of the vessel; and
determining at least one of vessel compliance and vessel segmentation as a function of the pulse transit time or pulse wave velocity and the applied pressure.

2. The method according to claim 1 wherein a fluid in the vessel is blood.

3. The method according to claim 1, wherein applying the series of pressure values to the region of a vessel comprises increasing pressure from zero to a suprasystolic pressure and then returning the pressure to zero.

4. The method according to claim 1, wherein applying the series of pressure values to a region of a vessel comprises inflating an inflatable cuff around the vessel.

5. The method according to claim 1, wherein the series of pressure values comprises a linear change in pressure resulting in a linear increase in the at least one of the pulse transit time and the pulse wave velocity.

6. The method according to claim 5, wherein determining at least one of compliance from the at least one of the pulse transit time and the pulse wave velocity comprises:
determining a change in the pulse transit time or the pulse wave velocity at each of the pressure values.

7. The method according to claim 5, wherein determining vessel segmentation from the at least one of the pulse transit time and the pulse wave velocity comprises:
determining regions of discontinuity in the at least one of the pulse transit time and the pulse wave velocity versus pressure change, wherein the regions of discontinuity indicate the arterial segmentation in the region of the vessel.

8. The method according to claim 7, additionally comprising plotting the at least one of the pulse transit time and the pulse wave velocity versus pressure change.

9. The method according to claim 1, additionally comprising:
incorporating an impedance sensor in combination with an inflatable cuff pressure generator; and
noninvasively applying the inflatable cuff pressure generator to the region of the vessel.

10. An apparatus for noninvasively obtaining a physiological parameter of a fluid vessel according to claim 1, the apparatus comprising:
a pressure applicator for applying external pressure to the local measurement area; and
an impedance measurer coextensive with the pressure applicator.

11. The method according to claim 1, wherein the applied pressure comprises an externally applied pressure of the vessel.

12. The method according to claim 1, wherein the applied pressure comprises an internally applied pressure of the vessel.

13. The method according to claim 1, additionally comprising calculating a static or steady state fluid volume of the region of the vessel.
